(19) United States
(12) Patent Application Publication
(10) Pub. No.: US 2020/0188278 A1
(43) Pub. Date: Jun. 18, 2020

(54) COMPOSITION AND METHOD FOR TEMPORARILY RESHAPING KERATINOUS FIBRES

(71) Applicant: Henkel AG & Co. KGaA, Duesseldorf (DE)

(72) Inventors: JULIA BIBIANE LANGE, Bad Bramstedt (DE); CYRIELLE MARTINEZ, Hamburg (DE); DIANE METTEN, Hamburg (DE)

(73) Assignee: Henkel AG & Co. KGaA, Duesseldorf (DE)

(21) Appl. No.: 16/718,048

(22) Filed: Dec. 17, 2019

(30) Foreign Application Priority Data
Dec. 18, 2018 (DE) 10 2018 222 037.9

(51) Int. Cl. A61K 8/73; A61K 8/81; A61Q 5/06

(52) U.S. Cl. CPC A61K 8/737; A61Q 5/06; A61K 8/817; A61K 8/8182

(57) ABSTRACT

A cosmetic composition for temporarily reshaping keratinous fibres, containing:

a) a cationically modified guar derivative with a weight average molecular weight in the range from about 5,000 to about 200,000 and a degree of cationic substitution in the range from about 0.1 to about 2 and

b) at least one copolymer comprising b1) quaternised vinylimidazole and b2) vinylpyrrolidone as monomers,

use thereof and method using such a composition.

### CROSS-REFERENCE TO RELATED APPLICATION

This application claims priority to German Patent Application No. 10 2018 222 037.9, filed Dec. 18, 2018, which is incorporated herein by reference in its entirety.

### TECHNICAL FIELD

The present disclosure relates to a cosmetic composition based on two selected polymers for setting hair or for temporary reshaping of keratinous fibres, in particular human hair, and methods making use of this composition and use thereof.

### BACKGROUND

The temporary shaping of hairstyles for a prolonged period lasting for up to several days typically requires the application of active setting ingredients. For this reason, hair treatment products which serve to lend hair a temporary shape have an important part to play. Corresponding compositions for temporary reshaping usually contain synthetic polymers and/or waxes as the active setting ingredient. Compositions to support the temporary reshaping of keratin-containing fibres may be packaged for example as hairspray, hair wax, hair gel or hair mousse.

The most important property of a composition for the temporary reshaping of hair, also described hereafter as styling products, consists in providing the treated hair in the newly shaped form—i.e., in a form imposed on the hair—the strongest hold possible. This is also described as a strong style hold or high hold strength of the styling product. The style hold is substantially determined by the nature and quantity of the active setting ingredient used, although the other constituents of the styling product may also have some influence.

Apart from a high hold strength, styling products must also satisfy a whole range of other requirements. These may be divided roughly into properties on the hair, properties of the respective formulation, e.g., properties of the mousse, the gel or the sprayed aerosol, and properties relating to the handling of the styling product, the properties on the hair being particularly important. Particularly notable among such properties are resistance to humidity, low stickiness (tack) and a balanced conditioning effect. Moreover, as far as possible a styling product should be universally usable for all hair types and mild on the hair and skin.

The hairstyle hold generally, and in the case of wavy hair “curl retention” are particular requirements that styling products are expected to satisfy. In this context, curl retention is a measure of the degree to which hair curls are retained. Curl retention is usually poorer when the treated hair is exposed to a humid environment, as the tendency of the hair to absorb moisture, that is to say water, reduces its ability to hold curls.

In order to satisfy the various requirements, many synthetic polymers have already been developed as active setting ingredients which are used in styling products. The polymers may be divided into cationic, anionic, non-ionic and amphoteric setting polymers.

Polymers with INCI designation Polyquaternium-16 are described in the field of hair care, particularly in hairsprays, are described in EP 1504744 B1.

### BRIEF SUMMARY

One object of the present disclosure was to make further suitable polymer combinations available, which are notable for their good film-forming and/or setting properties, have high hold strength without sacrificing flexibility and good resistance to humidity—in particular resistance to sweat and water. One object in particular of the present disclosure is to provide styling products of such kind that offer both good long-term hold and a high degree of curl retention in humid environments.

This was achieved as contemplated herein using a combination of two selected polymers.

The following are made possible by the present disclosure:

A cosmetic composition for temporarily reshaping keratinous fibres, containing:
a) at least one cationically modified guar derivative with a weight average molecular weight in the range from about 5,000 to about 200,000 and a degree of cationic substitution in the range from about 0.1 to about 2 and
b) at least one copolymer comprising b1) quaternised vinylimidazole and b2) vinylpyrrolidone as monomers.

The cosmetic composition as contemplated herein, wherein the weight of the cationically modified guar derivative a) constitutes from about 0.1 to about 10 wt %, preferably from about 0.15 to about 5 wt % and in particular from about 0.2 to about 2.5 wt % of the total weight of the composition.

The cosmetic composition as contemplated herein, wherein the cationically modified guar derivative a) has a weight average molecular weight in the range from about 20,000 to about 150,000, more preferably in the range from about 35,000 to about 100,000 and most particularly preferably in the range from about 50,000 to about 70,000.

The cosmetic composition as contemplated herein, wherein das cationically modified guar derivative a) has a degree of cationic substitution in the range from about 0.2 to about 1.

The cosmetic composition as contemplated herein, wherein the cationically modified guar derivative a) is selected from the group of compounds with the INCI designation Guar Hydroxypropyltrimonium Chloride.

The cosmetic composition as contemplated herein, wherein the weight of the copolymer b) constitutes from about 0.1 to about 10 wt %, particularly preferably from about 0.25 to about 8 wt % and particularly from about 0.5 to about 5 wt % of the total weight of the composition.

The cosmetic composition as contemplated herein, wherein the copolymer b) is obtained by reacting at least one monomer (b1) from the group of quaternised vinylimidazoles with N-Vinylpyrrolidone (b2).

The cosmetic composition as contemplated herein, wherein the copolymer b) is selected from the group of compounds with INCI designation Polyquaternium-16.

The cosmetic composition as contemplated herein, wherein the weight ratio between cationically modified guar derivative a) and copolymer b) is from about 10:1 to about 1:10, preferably from about 1:3 to about 1:7, and particularly preferably from about 1:5 to about 1:6.

The cosmetic composition as contemplated herein, wherein it further contains
c) Polyvinylpyrrolidone and/or vinylpyrrolidone/vinylacetate copolymer, preferably polyvinylpyrrolidone.

The cosmetic composition as contemplated herein, wherein the polyvinylpyrrolidone and/or vinylpyrrolidone/vinylacetate copolymer c) constitutes from about 0.1 to about 10 wt %, preferably from about 2 to about 8.5 wt % and particularly from about 3 to about 7 wt % of the total weight of the cosmetic composition.

The cosmetic composition as contemplated herein, wherein the composition contains from about 0.01 to about 5 wt %, preferably from about 0.02 to about 4 wt % and particularly from about 0.05 to about 2 wt % of an organic acid or salt thereof, preferably lactic acid or salt thereof, relative to its total weight.

The cosmetic composition as contemplated herein, wherein the cosmetic composition contains from about 0.01 to about 5 wt %, more preferably from about 0.01 to about 2 wt % and particularly preferably from about 0.02 to about 1.5 wt % of an alkanol amine or a neutralised form thereof, in particular 2-Amino-2-methylpropanol or a neutralised form thereof, relative to its total weight.

The cosmetic composition as contemplated herein, wherein the composition contains at least about 20 wt %, preferably at least about 40 wt % and particularly at least about 65 wt % water relative to its total weight.

The cosmetic composition as contemplated herein, wherein the composition is present in the form of a hair gel, hairspray, hair mousse, hair lotion or hair wax.

Use of a cosmetic composition as contemplated herein for temporarily reshaping keratin-containing fibres, in particular human hair.

Use of a cosmetic composition as contemplated herein for improving resistance to moisture of temporarily reshaped keratinous fibres.

Use of a cosmetic as contemplated herein for improving the degree of curl retention in a humid environment of temporarily reshaped keratinous fibres.

A method for temporarily reshaping keratinous fibres, in particular human hair, in which a cosmetic composition as contemplated herein is applied to the keratinous fibres, the shape of which is fixed temporarily.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Cationic guar derivatives are used in hair care products for grooming the hair, to lend the hair improved combability, for example. Cationic guar derivatives in hair care products can impart conditioning effects to the skin. In detergent and fabric softener formulations, cationic guar derivatives impart conditioning, softening, abrasion resistant and antistatic properties to the fabrics that are treated with them.

It was therefore the more surprising to discover that when selected cationic guar derivatives are combined with a setting/film-forming styling polymer, copolymer b), which is already used in styling products, it is possible to obtain outstanding hold in styling products. Other typically required properties of styling products, such as low tack, were retained. Such a good combination of properties was not to be expected, and was surprising even though the individual components were well known. It was further found that the combination of the two components resulted in a strongly super-additive, that is to say synergistic effect in terms of resistance to moisture, particularly in terms of the degree of curl retention in a humid environment, which manifested itself in the HHCR test (High Humidity Curl Retention Test).

For the purposes of the present disclosure, the term keratinous fibres includes furs, wool and feathers, but particularly human hair. In this context, human hair may include hair on the head and/or facial hair.

The essential constituents of the cosmetic composition are the cationic guar derivative a) and the copolymer b), comprising b1) quaternised vinylimidazole and b2) vinylpyrrolidone as monomers.

The cosmetic compositions are exemplified in particular by an improved degree of curl retention in a humid environment compared with alternative cosmetic compositions. A weight ratio between the cationically modified guar derivative a) and the copolymer b) in the cosmetic composition from about 10:1 to about 1:10, preferably from about 1:3 to about 1:7 and particularly preferably from about 1:5 to about 1:6 has proven to be particularly favourable for the cosmetic properties of the compositions.

The cosmetic compositions contain a cationic guar derivative a) as the first essential component.

In the context of this application, the term “guar derivatives” is understood to include (bio)chemically and/or physically modified guar gums. Guar gum is a polysaccharide composed of galactose and mannose which has a linear backbone of β-1,4-linked mannose residues. Galactose residues are linked to every second mannose residue in this backbone via β-1,6 glycosidic bonds. These guar gums may be modified chemically for example by esterification or etherification of the hydroxy groups in the polysaccharide or by reacting with alkalis, acids or oxidants. These guar gums may be modified biochemically for example by reacting with hydrolytic enzymes, bacteria or fungi. A physical modification is possible for example using heat, radiation, and comminution with the aid of a high-speed stirrer, for example.

The term “cationically modified guar derivatives” are understood to be guar gums whose hydroxy groups have been esterified or etherified with a compound that includes at least one cationic group. This cationic group may be either permanently cationic or temporarily cationic. Compounds are considered to be “permanently cationic” if they include a cationic group regardless of the pH value of the cosmetic composition. These include in particular compounds with quaternary nitrogen atoms, such as quaternary ammonium groups. On the other hand, compounds which only contain a cationic group for certain pH values, particularly pH values in the acidic range, are described as “temporarily cationic”. Examples of temporarily cationic groups are amine groups.

There are very many different methods for adding the cationic functionality. Thus for example the starter material may be reacted for long enough and at a sufficiently high temperature with a tertiary amine compound or a quaternary amine compound containing groups which are capable of reacting with reactive groups of the guar, in particular the hydroxy groups.

Compounds that are suitable for introducing the cationic functionality include for example 2-Dialkylaminoethyl chloride and quaternary ammonium compounds such as 3-Chloro-2-hydroxypropyltrimethylammonium chloride and 2,3-Epoxypropyltrimethylammonium chloride. Further examples are glycidyltrialkylammonium salts and 3-Halogen-2-hydroxypropyltrialkylammonium salts such as glycidyltrimethylammonium chloride, glycidyltriethylammonium chloride, gylcidyltripropylammonium chloride, glycidylethyldimethylammonium chloride, glycidyldiethylmethylammonium chloride, and the corresponding bromides and iodides; 3-Chloro-2-hydroxypropyltrimethylammonium chloride, 3-Chloro-2-hydroxypropyltriethylammonium chloride, 3-Chloro-2-hydroxypropyltripropylammonium chloride, 3-Chloro-2-hydroxypropylethyldimethylammonium chloride and the corresponding bromides and iodides; and quaternary ammonium compounds such as halides of compounds containing an imidazole ring.

It is preferred that the cationically modified guar derivative a) comprises at least one structural unit with the formula (I),

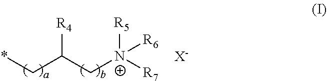

in which
R₄ stands for hydrogen, a C₁₋₄ alkyl group or a hydroxyl group,
R₅, R₆ and R₇ each stand independently of one another for a C₁₋₈ alkyl group,
a and b each stand independently of one another for integers from 1 to 3,
X- stands for a physiologically compatible anion.

Examples of C₁₋₄ alkyl groups are methyl-, ethyl-, propyl-, isopropyl-, butyl-, sec-butyl-, isobutyl- or tert-butyl groups.

Examples of C₁₋₈ alkyl groups are methyl-, ethyl-, propyl-, isopropyl-, butyl-, sec-butyl-, isobutyl-, tert-butyl-, pentyl- and hexyl-, heptyl- and octyl groups.

The radical R₄ in the structural unit of formula (I) preferably stands for a hydroxyl group, and a and b each stand independently of one another for the integer 1.

It is further preferable that the radicals R₅ to R₇ in the structural unit of formula (I) each stand independently of one another for a C₁₋₆ alkyl group, preferably for a C₁₋₄ alkyl group, more preferably for a C₁₋₃ alkyl group in particular for a C₁ alkyl group, and X⁻ stands for a halide ion, in particular chloride.

Other derivatisations of the cationic guar derivative with non-ionic substituents, that is to say hydroxyalkyl, in which the alkyl represents a straight or branched hydrocarbon radical with 1 to 6 carbon atoms (e.g., hydroxyethyl, hydroxypropyl, hydroxybutyl), or anionic substituents such as carboxymethyl groups, are optional. These optional substituents may be added to the cationic guar derivative by reacting with reagents such as (1) alkylene oxides (e.g., ethylene oxide, propylene oxide, butylene oxide) to obtain hydroxyethyl groups, hydroxypropyl groups or hydroxybutyl groups, or with (2) chloromethyl acetic acid to obtain a carboxymethyl group. However, it is extremely preferable if the cationic guar derivative a) contains no other substituents of either non-ionic or anionic nature.

The cationically modified guar derivative a) has a weight average molecular weight in the range from about 5,000 to about 200,000. It is preferable that the cationically modified guar derivative a) have a weight average molecular weight in the range from about 20,000 to about 150,000, more preferably in the range from about 35,000 to about 100,000, and most particularly preferably in the range from about 50,000 to about 70,000.

The weight average molecular weight can be determined for example by employing gel permeation chromatography using a polystyrene standard.

The cationically modified guar derivative a) has a degree of cationic substitution (DS) in the range from about 0.1 to about 2. It is preferable if the degree of cationic substitution is in the range from about 0.2 to about 1.

It is particularly advantageous if the cationically modified guar derivative a) has a degree of substitution (DS) by the structural unit of formula (I) from about 0.1 to about 2, in particular from about 0.2 to about 1.

The degree of substitution (DS) describes the average number of cationic structure units, in particular cationic structure units of formula (I), which are bound for each monomer of the polysaccharide, that is to say per anhydromannose and anhydrogalactose. Since each monomer of the polysaccharide includes on average about 3 free OH groups, the DS may have values between 0 and about 3. Thus for example a DS value of about 1 means that on average one cationic structure unit is bound per monomer of the polysaccharide, and consequently each monomer still has about 2 free OH groups. The degree of substitution (DS) may be determined by ¹H-NMR spectroscopy or titration, for example.

It is extremely preferable that the cationically modified guar derivative comprise a cationically modified guar derivative with the INCI designation “Guar Hydroxypropyltrimonium Chloride”, which has a weight average molecular weight in the range from about 5,000 to about 200,000 and a degree of cationic substitution in the range from about 0.1 to about 2. Such a cationically modified guar derivative is available commercially from Ashland Specialty Chemical for example with the name “N-Hance CCG 45 Cationic Guar”.

The cationic guar derivative a) preferably constitutes from about 0.1 to about 10 wt %, particularly preferably from about 0.15 to about 5 wt % and particularly from about 0.2 to about 2.5 wt % of the total weight of the composition.

As the second essential constituent, the cosmetic compositions contain at least one copolymer b) which comprises b1) quaternised vinylimidazole and b2) vinylpyrrolidone as monomers. Preferred copolymers b) have no thickening effect.

A particularly preferred embodiment of cosmetic compositions is the copolymer b) is selected from the group of compounds with the INCI designation Polyquaternium-16.

Polyquaternium-16 compounds are copolymers that can also be designated as Poly[(3-methyl-1-vinylimidazolium chloride)-co-(1-vinylpyrrolidone)].

Such copolymers are available commercially from BASF under the trade name Luviquat, for example:

| | Solid | | | |
|---|---|---|---|---|
| | content | Molar | Ratio | Charge density |
| Trade name | % | mass | VP/QVI | (mEq/g) |
| Luviquat | 38-42 | 40,000 | 5/95 | 6.1 |
| Excellence | | | | |
| Luviquat FC | 38-42 | 80,000 | 50/50 | 2.0 |
| 550 | | | | |
| Luviquat FC | 38-42 | 100,000 | 70/30 | 3.3 |
| 370 | | | | |
| Luviquat Style | 19-21 | 400,000 | 55/45 | 3.0 |

The copolymers differ in respect of the ratio of vinylpyrrolidone (VP) to quaternised vinylimidazole (QVI), their molar mass and/or their charge density.

The use of copolymers b) with a monomer ratio between vinylpyrrolidone (VP) and quaternised vinylimidazole (QVI) in the range from about 30 to about 70% (VP) to from about 70 to about 30% (QVI) and a molar mass in the range from about 50,000 to about 150,000 Da proved to be particularly advantageous for solving the problem.

The use of a copolymer b) with a monomer ratio between vinylpyrrolidone (VP) and quaternised vinylimidazole (QVI) in the range from about 70 to about 30% (QVI) and a molar mass of about 100,000 Da proved to be extremely preferred.

The copolymer b) constitutes preferably from about 0.1 to about 10 wt %, particularly preferably from about 0.25 to about 8 wt % and particularly from about 0.5 to about 5 wt % of the total weight of the composition.

It may be preferred that the cosmetic composition contain one or more further polymer(s) which is/are different from the polymers a) and b), and for example enhance the effect of the thickening agents or gel formation or film formation. Examples are cationic, anionic, non-ionic or amphoteric polymers.

Examples are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6 Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polyquaternium-68, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer.

The further component acting as a gelling agent is preferably a homopolyacrylic acid (INCI: Carbomer), which is commercially available for example under the name Carbopol® in various versions. The carbomer is preferably contained in a proportion from about 0.02 to about 3 wt %, preferably from about 0.05 to about 1.5 wt %, more preferably still from about 0.2 to about 0.8 wt % relative to the total weight of the cosmetic composition.

In order to increase their cosmetic effect further, besides the polymers a) and b) and an optionally added thickening agent or gelling agent, preferred compositions also contain a film-forming polymer c) which is not the same as the abovementioned substances, and in particular contains an anionic or non-ionic polymer c).

Examples of non-ionic polymers are:
vinylpyrrolidone/vinyl ester copolymers, as they are marketed for example under the trade name Luviskol (BASF). Luviskol VA 64 and Luviskol VA 73, each being vinylpyrrolidone/vinyl acetate copolymers, are preferred non-ionic polymers.
cellulose ethers, such as hydroxypropyl cellulose, hydroxyethyl cellulose, and methyl hydroxypropyl cellulose, as they are marketed for example, under the trade names Culminal and Benecel (AQUALON).
shellac.
polyvinylpyrrolidones, as they are marketed for example under the trade name Luviskol (BASF).
siloxanes. These siloxanes may be either water-soluble or water-insoluble. Both volatile and non-volatile siloxanes are suitable, non-volatile siloxanes being understood to be those compounds whose boiling point at normal pressure is above about 200° C. Preferred siloxanes are polydialkylsiloxanes such as, for example, polydimethylsiloxane, polyalkylarylsiloxanes such as, for example, polyphenylmethylsiloxane, ethoxylated polydialkylsiloxanes, and polydialkylsiloxanes which include amine and/or hydroxy groups.
glycosidically substituted silicones.

Film-forming polymers that are preferred for use due to their cosmetic effect in combination with the copolymers a) and b) are Polyvinylpyrrolidone (INCI designation: PVP) and the Vinylpyrrolidone/Vinylacetate copolymers (INCI designation VP/VA Copolymer). The hold properties and also the application properties of the cosmetic compositions are distinctly improved by the addition of film-forming polymers, particularly the abovementioned polyvinylpyrrolidones and vinylpyrrolidone/vinylacetate copolymers. The percentage by weight of these polymers is preferably limited to quantities between about 1.0 and about 10 wt %. Preferred cosmetic compositions further contain from about 1 to about 10 wt % polyvinylpyrrolidone and/or vinylpyrrolidone/vinylacetate copolymer, preferably polyvinylpyrrolidone, relative to their total weight. Particularly preferred cosmetic compositions contain the polyvinylpyrrolidone and/or vinylpyrrolidone/vinylacetate copolymer c) in a weight percentage from about 2 to about 8.5 wt %, preferably from about 3 to about 7 wt % of the total weight of the cosmetic composition.

The cosmetic composition may contain further usual ingredients of styling products. Additional care substances may be noted in particular as further suitable excipients and additives.

For example, the composition may contain at least one protein hydrolysate and/or one of its derivatives as the care product. Protein hydrolysates are product mixtures which are obtained by acidically, basically or enzymatically catalysed degradation of proteins. The term protein hydrolysates is also understood to include total hydrolysates and individual amino acids and derivatives thereof as well as mixtures of various amino acids.

The composition may further contain at least one vitamin, one provitamin, one vitamin precursor and/or or one derivative thereof as the care product. In this context, those vitamins, provitamins and vitamin precursors which are typically assigned to the groups A, B, C, E, F and H are preferred.

Similarly to the addition of glycerol and/or propylene glycol, the addition of panthenol increases the flexibility of the polymer film that is formed when the composition is applied.

The compositions may further contain at least one plant extract, but also mono- or oligosaccharides and/or lipids as the care product.

Oleosomes are also suitable for use as the care product. The natural and synthetic cosmetic oleosomes include for example plant oils, liquid paraffin oils, isoparaffin oils and synthetic hydrocarbons as well as di-n-alkyl ethers having a total of between 12 and 36 C atoms, in particular 12 to 24 C atoms. Preferred cosmetic compositions contain at least one oleosome, preferably at least one oleosome from the group of silicone oils. The group of silicone oils includes in particular the dimethicones, which further comprises the cyclomethicones, the aminofunctional silicones and the dimethiconols. The dimethicones may be either linear or branched or cyclic or cyclic and branched. Suitable silicone oils or silicone gums are in particular dialkyl- and alkylaryl siloxanes, such as for example dimethyl polysiloxane and methylphenyl polysiloxane, and the alkoxylated, quaternised or also anionic derivatives thereof. Preferred are cyclic and linear polydialkyl siloxanes, the alkoxylated and/or aminated derivatives thereof, dihydroxy polydimethyl siloxanes and polyphenylalkyl siloxanes.

Further preferred oil-containing care components are ester oils, i.e., esters of C6-C30 fatty acids with C2-C30 fatty alcohols, preferably monoesters of fatty acids with alcohols having 2 to 24 C atoms, such as, for example, isopropyl myristate (Rilanit® IPM), isononanoic acid C16-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V).

Also suitable as care products are dicarboxylic acid esters, symmetric, asymmetric, or cyclic esters of carbonic acid with fatty alcohols, tri-fatty acid esters of saturated and/or unsaturated, linear and/or branched fatty acids with glycerol, or fatty acid partial glycerides, which are understood to be monoglycerides, diglycerides, and technical mixtures thereof.

The composition preferably also includes emulsifiers or surface-active agents. Preferred are PEG derivatives of hydrogenated castor oil, which are commercially available e.g., under the name PEG Hydrogenated Castor Oil, e.g., PEG-30 Hydrogenated Castor Oil, PEG-33 Hydrogenated Castor Oil, PEG-35 Hydrogenated Castor Oil, PEG-36 Hydrogenated Castor Oil or PEG-40 Hydrogenated Castor Oil. The use of PEG-40 Hydrogenated Castor Oil is preferred. These are preferably contained in a quantity from about 0.05 to about 1.5 wt %, more preferably from about 0.1 to about 1 wt %, also preferably from about 0.2 to about 0.8 wt % or from about 0.3 to about 0.6 wt %. The addition of the surface-active agents, particularly the aforementioned PEG derivatives of hydrogenated castor oil, has the effect of making the cosmetic compositions not only easier to package but also easier to wash out.

The cosmetic compositions contain the constituents or active ingredients in a cosmetically acceptable carrier.

Preferred cosmetically acceptable carriers are aqueous, alcoholic or aqueous-alcoholic media containing preferably at least about 10 wt % water calculated for the total weight of the composition. The cosmetic carrier particularly preferably contains water in particular in such a quantity that the cosmetic composition contains at least about 20 wt %, particularly at least about 40 wt %, most preferably at least about 65 wt % water relative to its total weight. Most particularly preferred cosmetic compositions have a water component from about 50 to about 95 wt %, preferably from about 60 to about 90 wt % and in particular from about 65 to about 85 wt % relative to their total weight.

Particularly the lower alcohols with 1 to 4 carbon atoms normally used for cosmetic purposes, such as ethanol and isopropanol, may be included as alcohols.

Examples of water-soluble solvents as cosolvent are glycerol and/or ethylene glycol and/or 1,2-Propylene glycol in a quantity from 0 to about 30 wt % relative to the total composition.

It may be preferable for the composition to contain an organic acid or salt thereof. The organic acid is preferably selected from the group including maleic acid, lactic acid, acetic acid, propane acid, citric acid, tartaric acid, succinic acid, oxalic acid, gluconic acid, malic acid, amino acids and mixtures thereof. The organic acid most particularly preferably includes lactic acid.

The organic acid or its salt, preferably lactic acid or its salt, preferably constitutes from about 0.01 to about 5 wt %, more preferably from about 0.02 to about 4 wt %, and particularly preferably from about 0.05 to about 2 wt % of the total weight of the cosmetic composition.

The cationically modified guar derivative is preferably introduced in the form of an acidic, aqueous solution. An organic acid is preferably used to acidify the aqueous solution.

It may be preferred that the cosmetic composition further contains an alkanolamine. The alkanolamines that are usable as alkalisation agents are preferably selected from primary amines with a C₂-C₆ alkyl base body supporting at least one hydroxyl group. Particularly preferred alkanolamines are selected from the group including 2-Aminoethan-1-ol (monoethanolamine), Tris(2-hydroxyethyl)-amine (triethanolamine), 3-Aminopropan-1-ol, 4-Aminobutan-1-ol, 5-Aminopentan-1-ol, 1-Aminopropan-2-ol, 1-Aminobutan-2-ol,1-Aminopentan-2-ol, 1-Aminopentan-3-ol, 1-Aminopentan-4-ol, 3-Amino-2-methylpropan-1-ol, 1-Amino-2-methylpropan-2-ol, 3-Aminopropan-1,2-diol, 2-Amino-2-methylpropan-1,3-diol. Most particularly preferred alkanolamines are selected from the group of 2-Aminoethan-1-ol, 2-Amino-2-methylpropan-1-ol and 2-Amino-2-methyl-propan-1,3-diol. 2-Amino-2-methylpropanol has proven to be particularly suitable. The aminoalcohol or its neutralised form, preferably 2-Amino-2-methylpropanol, preferably constitutes a percentage by weight from about 0.01 to about 5 wt %, more preferably from about 0.01 to about 2 wt % and particularly preferably from about 0.02 to about 1.5 wt % of the total weight of the cosmetic composition.

The constitutions of some preferred cosmetic compositions are listed in the following tables (unless otherwise indicated, quantities in wt % relative to the total weight of the cosmetic composition).

| | Formula | Formula | Formula | Formula | Formula |
|---|---|---|---|---|---|
| | 1 | 2 | 3 | 4 | 5 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Misc | to 100 | to 100 | to 100 | to 100 | to 100 |

| | Formula | Formula | Formula | Formula | Formula |
|---|---|---|---|---|---|
| | 1a | 2a | 3a | 4a | 5a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Misc | to 100 | to 100 | to 100 | to 100 | to 100 |

| | Formula | Formula | Formula | Formula | Formula |
|---|---|---|---|---|---|
| | 1b | 2b | 3b | 4b | 5b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| Misc | to 100 | to 100 | to 100 | to 100 | to 100 |
| | Formula | Formula | Formula | Formula | Formula |
| | 1c | 2c | 3c | 4c | 5c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| Misc | to 100 | to 100 | to 100 | to 100 | to 100 |

| | Formula | Formula | Formula | Formula | Formula |
|---|---|---|---|---|---|
| | 6 | 7 | 8 | 9 | 10 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Polyvinylpyrrolidone | 1 to 10 | 2 to 8.5 | 2 to 8.5 | 3 to 7 | 3 to 7 |
| Misc | to 100 | to 100 | to 100 | to 100 | to 100 |
| | Formula | Formula | Formula | Formula | Formula |
| | 6a | 7a | 8a | 9a | 10a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Polyvinylpyrrolidone | 1 to 10 | 2 to 8.5 | 2 to 8.5 | 3 to 7 | 3 to 7 |
| Misc | to 100 | to 100 | to 100 | to 100 | to 100 |
| | Formula | Formula | Formula | Formula | Formula |
| | 6b | 7b | 8b | 9b | 10b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| Polyvinylpyrrolidone | 1 to 10 | 2 to 8.5 | 2 to 8.5 | 3 to 7 | 3 to 7 |
| Misc | to 100 | to 100 | to 100 | to 100 | to 100 |
| | Formula | Formula | Formula | Formula | Formula |
| | 6c | 7c | 8c | 9c | 10c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| Polyvinylpyrrolidone | 1 to 10 | 2 to 8.5 | 2 to 8.5 | 3 to 7 | 3 to 7 |
| Misc | to 100 | to 100 | to 100 | to 100 | to 100 |

| | Formula | Formula | Formula | Formula | Formula |
|---|---|---|---|---|---|
| | 11 | 12 | 13 | 14 | 15 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Vinylpyrrolidone/Vinylacetate | 1 to 10 | 2 to 8.5 | 2 to 8.5 | 3 to 7 | 3 to 7 |
| copolymer | | | | | |
| Misc | to 100 | to 100 | to 100 | to 100 | to 100 |
| | Formula | Formula | Formula | Formula | Formula |
| | 11a | 12a | 13a | 14a | 15a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Vinylpyrrolidone/Vinylacetate | 1 to 10 | 2 to 8.5 | 2 to 8.5 | 3 to 7 | 3 to 7 |
| copolymer | | | | | |
| Misc | to 100 | to 100 | to 100 | to 100 | to 100 |
| | Formula | Formula | Formula | Formula | Formula |
| | 11b | 12b | 13b | 14b | 15b |
| N-Hance CCG 45 (specified | 0.1 to 10 | 0.15 to 5 | 0.15 to 5 | 0.2 to 2.5 | 0.2 to 2.5 |
| as solid content) | | | | | |
| Luviquat FC 370 (specified | 0.1 to 10 | 0.25 to 8 | 0.25 to 8 | 0.5 to 5 | 0.5 to 5 |
| as solid content) | | | | | |
| Vinylpyrrolidone/Vinylacetate | 1 to 10 | 2 to 8.5 | 2 to 8.5 | 3 to 7 | 3 to 7 |
| copolymer | | | | | |
| Misc | to 100 | to 100 | to 100 | to 100 | to 100 |
| | Formula | Formula | Formula | Formula | Formula |
| | 11c | 12c | 13c | 14c | 15c |
| N-Hance CCG 45 (specified | 0.1 to 10 | 0.15 to 5 | 0.15 to 5 | 0.2 to 2.5 | 0.2 to 2.5 |
| as solid content) | | | | | |
| Luviquat FC 550 (specified | 0.1 to 10 | 0.25 to 8 | 0.25 to 8 | 0.5 to 5 | 0.5 to 5 |
| as solid content) | | | | | |
| Vinylpyrrolidone/Vinylacetate | 1 to 10 | 2 to 8.5 | 2 to 8.5 | 3 to 7 | 3 to 7 |
| copolymer | | | | | |
| Misc | to 100 | to 100 | to 100 | to 100 | to 100 |

| | Formula | Formula | Formula | Formula | Formula |
|---|---|---|---|---|---|
| | 16 | 17 | 18 | 19 | 20 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Carbomer | 0.02 to 3 | 0.05 to 2 | 0.05 to 1.5 | 0.2 to 1.5 | 0.2 to 0.8 |
| Misc | to 100 | to 100 | to 100 | to 100 | to 100 |

| | Formula | Formula | Formula | Formula | Formula |
|---|---|---|---|---|---|
| | 16a | 17a | 18a | 19a | 20a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Carbomer | 0.02 to 3 | 0.05 to 2 | 0.05 to 1.5 | 0.2 to 1.5 | 0.2 to 0.8 |
| Misc | to 100 | to 100 | to 100 | to 100 | to 100 |

| | Formula | Formula | Formula | Formula | Formula |
|---|---|---|---|---|---|
| | 16b | 17b | 18b | 19b | 20b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| Carbomer | 0.02 to 3 | 0.05 to 2 | 0.05 to 1.5 | 0.2 to 1.5 | 0.2 to 0.8 |
| Misc | to 100 | to 100 | to 100 | to 100 | to 100 |
| | Formula | Formula | Formula | Formula | Formula |
| | 16c | 17c | 18c | 19c | 20c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| Carbomer | 0.02 to 3 | 0.05 to 2 | 0.05 to 1.5 | 0.2 to 1.5 | 0.2 to 0.8 |
| Misc | to 100 | to 100 | to 100 | to 100 | to 100 |

| | Formula | Formula | Formula | Formula | Formula |
|---|---|---|---|---|---|
| | 21 | 22 | 23 | 24 | 25 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | to 100 | to 100 | to 100 | to 100 | to 100 |
| | Formula | Formula | Formula | Formula | Formula |
| | 21a | 22a | 23a | 24a | 25a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | to 100 | to 100 | to 100 | to 100 | to 100 |
| | Formula | Formula | Formula | Formula | Formula |
| | 21b | 22b | 23b | 24b | 25b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | to 100 | to 100 | to 100 | to 100 | to 100 |
| | Formula | Formula | Formula | Formula | Formula |
| | 21c | 22c | 23c | 24c | 25c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | to 100 | to 100 | to 100 | to 100 | to 100 |

| | Formula | Formula | Formula | Formula | Formula |
|---|---|---|---|---|---|
| | 26 | 27 | 28 | 29 | 30 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 26a | 27a | 28a | 29a | 30a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 26b | 27b | 28b | 29b | 30b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 26c | 27c | 28c | 29c | 30c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 31 | 32 | 33 | 34 | 35 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 31a | 32a | 33a | 34a | 35a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 31b | 32b | 33b | 34b | 35b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 31c | 32c | 33c | 34c | 35c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 36 | 37 | 38 | 39 | 40 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Vinylpyrrolidone/Vinylacetate | 1 to 10 | 2 to 8.5 | 2 to 8.5 | 3 to 7 | 3 to 7 |
| copolymer | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 36a | 37a | 38a | 39a | 40a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Vinylpyrrolidone/Vinylacetate | 1 to 10 | 2 to 8.5 | 2 to 8.5 | 3 to 7 | 3 to 7 |
| copolymer | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 36b | 37b | 38b | 39b | 40b |
| N-Hance CCG 45 (specified | 0.1 to 10 | 0.15 to 5 | 0.15 to 5 | 0.2 to 2.5 | 0.2 to 2.5 |
| as solid content) | | | | | |
| Luviquat FC 370 (specified | 0.1 to 10 | 0.25 to 8 | 0.25 to 8 | 0.5 to 5 | 0.5 to 5 |
| as solid content) | | | | | |
| Vinylpyrrolidone/Vinylacetate | 1 to 10 | 2 to 8.5 | 2 to 8.5 | 3 to 7 | 3 to 7 |
| copolymer | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 36c | 37c | 38c | 39c | 40c |
| N-Hance CCG 45 (specified | 0.1 to 10 | 0.15 to 5 | 0.15 to 5 | 0.2 to 2.5 | 0.2 to 2.5 |
| as solid content) | | | | | |
| Luviquat FC 550 (specified | 0.1 to 10 | 0.25 to 8 | 0.25 to 8 | 0.5 to 5 | 0.5 to 5 |
| as solid content) | | | | | |
| Vinylpyrrolidone/Vinylacetate | 1 to 10 | 2 to 8.5 | 2 to 8.5 | 3 to 7 | 3 to 7 |
| copolymer | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 41 | 42 | 43 | 44 | 45 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 41a | 42a | 43a | 44a | 45a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 41b | 42b | 43b | 44b | 45b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 41c | 42c | 43c | 44c | 45c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 46 | 47 | 48 | 49 | 50 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 46a | 47a | 48a | 49a | 50a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 46b | 47b | 48b | 49b | 50b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 46c | 47c | 48c | 49c | 50c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 51 | 52 | 53 | 54 | 55 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 51a | 52a | 53a | 54a | 55a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 51b | 52b | 53b | 54b | 55b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 51c | 52c | 53c | 54c | 55c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

| | Formula | Formula | Formula | Formula | Formula |
|---|---|---|---|---|---|
| | 56 | 57 | 58 | 59 | 60 |
| Polymer a)* | 0.1 to 10 | 0.15 to 5 | 0.15 to 5 | 0.2 to 2.5 | 0.2 to 2.5 |
| Copolymer b) | 0.1 to 10 | 0.25 to 8 | 0.25 to 8 | 0.5 to 5 | 0.5 to 5 |
| Vinylpyrrolidone/Vinylacetate | 1 to 10 | 2 to 8.5 | 2 to 8.5 | 3 to 7 | 3 to 7 |
| copolymer | | | | | |
| PEG-40 Hydrogenated Castor | 0.05 to 1.5 | 0.1 to 1 | 0.2 to 0.8 | 0.3 to 0.8 | 0.3 to 0.6 |
| Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 56a | 57a | 58a | 59a | 60a |
| Guar Hydroxypropyl- | 0.1 to 10 | 0.15 to 5 | 0.15 to 5 | 0.2 to 2.5 | 0.2 to 2.5 |
| trimonium Chloride** | | | | | |
| Polyquaternium-16 | 0.1 to 10 | 0.25 to 8 | 0.25 to 8 | 0.5 to 5 | 0.5 to 5 |
| Vinylpyrrolidone/Vinylacetate | 1 to 10 | 2 to 8.5 | 2 to 8.5 | 3 to 7 | 3 to 7 |
| copolymer | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 56b | 57b | 58b | 59b | 60b |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 370 | 0.1 to 10 | 0.25 to 8 | 0.25 to 8 | 0.5 to 5 | 0.5 to 5 |
| (specified as solid | | | | | |
| content) | | | | | |
| Vinylpyrrolidone/Vinylacetate | 1 to 10 | 2 to 8.5 | 2 to 8.5 | 3 to 7 | 3 to 7 |
| copolymer | | | | | |
| PEG-40 Hydrogenated Castor | 0.05 to 1.5 | 0.1 to 1 | 0.2 to 0.8 | 0.3 to 0.8 | 0.3 to 0.6 |
| Oil | | | | | |
| Water | 50 to 95 | 50 to 95 | 60 to 90 | 60 to 90 | 65 to 85 |
| Misc | to 100 | to 100 | to 100 | to 100 | to 100 |
| | Formula | Formula | Formula | Formula | Formula |
| | 56c | 57c | 58c | 59c | 60c |
| N-Hance CCG 45 | 0.1 to 10 | 0.15 to 5 | 0.15 to 5 | 0.2 to 2.5 | 0.2 to 2.5 |
| (specified as solid | | | | | |
| content) | | | | | |
| Luviquat FC 550 | 0.1 to 10 | 0.25 to 8 | 0.25 to 8 | 0.5 to 5 | 0.5 to 5 |
| (specified as solid | | | | | |
| content) | | | | | |
| Vinylpyrrolidone/Vinylacetate | 1 to 10 | 2 to 8.5 | 2 to 8.5 | 3 to 7 | 3 to 7 |
| copolymer | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | to 100 | to 100 | to 100 | to 100 | to 100 |

cationically modified guar derivative with a weight average molecular weight from about 5,000 to about 200,000 and degree of cationic substitution (DS) from about 0.1 to about 2

with a weight average molecular weight from about 5,000 to about 200,000 and degree of cationic substitution (DS) from about 0.1 to about 2

“Misc” is understood to denote a cosmetic carrier, in particular (unless listed separately) water and optionally other usual constituents of styling products.

The cosmetic composition of the present disclosure may be prepared for delivery in the forms usually utilised for temporarily reshaping hair, e.g., as hair gel, hairspray, hair mousse, hair lotion or hair wax. It is preferably prepared in the form of a hair gel.

Both hair mousses and hairsprays require the presence of propellants. However, preferably no hydrocarbons or only small quantities thereof should be used for this. Propane, propane/butane mixtures and dimethyl ether are particularly suitable propellants.

The present disclosure also relates to the use of cosmetic compositions as contemplated herein for temporarily reshaping keratinous fibres, in particular human hair, and a method for temporarily reshaping keratinous fibres, in particular human hair, in which the keratinous fibres are exposed to a cosmetic composition as contemplated herein and temporarily set in the given shape.

The preceding notes on the cosmetic composition also apply mutatis mutandis to further preferred embodiments of the use and the method.

A further object of this patent application is the use of a cosmetic composition as contemplated herein to improve the degree of curl retention of temporarily reshaped keratinous fibres in a humid environment (HHCR).

### EXAMPLES

I. The following hair gels were produced:

| Component/Raw | INCI designation or | | | |
|---|---|---|---|---|
| material | chemical name | V1 | V2 | E1 |
| N-Hance CCG 45 ¹ | Guar | 1 | — | 0.5 |
| | Hydroxypropyltrimonium | | | |
| | Chloride | | | |
| Luviquat FC 550 ² | Polyquaternium-16 | — | 12.5 | 6.25 |
| Water | | 99 | 97.5 | 93.25 |
| Total | | 100 | 100 | 100 |

¹ 92 wt % active substance in water
² 38-42 wt % active substance in water

The quantities listed in the table represent the percentage by weight of the respective raw material relative to the total composition.

For the styling products obtained in this way, the degree of curl retention in humid environment was determined by employing a HHCR-Test (High Humidity Curl Retention-Test: 6 h) on cleaned Kerling hair strands (average from determination on 5 hair strands in each case):

| | V1 | V2 | E1 |
|---|---|---|---|
| HHCR | 19.4% | 26.7% | 35.1% |

According to the results, the polymer combination E1 as contemplated herein exhibited a clear super-additive, synergistic effect in terms of the degree of curl retention in humid environment.

II. The following hair gels were produced:

| Component/Raw | INCI designation or | | | |
|---|---|---|---|---|
| material | chemical name | V1 | V2 | E1 |
| N-Hance CCG 45 ¹ | Guar | 1 | — | 0.5 |
| | Hydroxypropyltrimonium | | | |
| | Chloride | | | |
| Luviquat FC 370 ² | Polyquaternium-16 | — | 12.5 | 6.25 |
| Water | | 99 | 97.5 | 93.25 |
| Total | | 100 | 100 | 100 |

¹ 92 wt % active substance in water
² 38-42 wt % active substance in water

The quantities listed in the table represent the percentage by weight of the respective raw material relative to the total composition.

For the styling products obtained in this way, the degree of curl retention in humid environment was determined by employing a HHCR-Test (High Humidity Curl Retention-Test: 6 h) on cleaned Kerling hair strands (average from determination on 5 hair strands in each case):

| | V1 | V2 | E1 |
|---|---|---|---|
| HHCR | 19.4% | 19.5% | 49.7% |

According to the results, the polymer combination E1 as contemplated herein exhibited a clear super-additive, synergistic effect in terms of the degree of curl retention in humid environment.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for temporarily reshaping keratinous fibres, comprising:
a) at least one cationically modified guar derivative with a weight average molecular weight in the range from about 5,000 to about 200,000 and a degree of cationic substitution in the range from about 0.1 to about 2 and
b) at least one copolymer comprising b1) quaternised vinylimidazole and b2) vinylpyrrolidone as monomers.

2. The cosmetic composition according to claim 1, wherein the weight of the cationically modified guar derivative a) constitutes from about 0.1 to about 10 wt % of the total weight of the composition.

3. The cosmetic composition according to claim 1, wherein the cationically modified guar derivative a) is selected from the group of compounds with the INCI designation Guar Hydroxypropyltrimonium Chloride.

4. The cosmetic composition according to claim 1, wherein the weight of the copolymer b) constitutes from about 0.1 to about 10 wt % of the total weight of the composition.

5. The cosmetic composition according to claim 1, wherein the copolymer b) is selected from the group of compounds with INCI designation Polyquaternium-16.

6. The cosmetic composition according to claim 1, wherein the copolymer b) has a monomer ratio between vinylpyrrolidone (VP) and quaternised vinylimidazole (QVI) in the range from about 30 to about 70% (VP) to 70 to 30% (QVI) and a molar mass in the range from about 50,000 to about 150,000 Da.

7. The cosmetic composition according to claim 1, characterized in that the composition further comprises an organic acid or salt thereof.

8. The cosmetic composition according to claim 1, wherein the composition comprises at least about 20 wt % water relative to its total weight.

9. Use of a cosmetic composition according to claim 1 for temporarily reshaping keratinous fibres.

10. A method for temporarily reshaping keratinous fibres, in which a cosmetic composition according to claim 1 is applied to the keratinous fibres, the shape of which is fixed temporarily thereby.
